(19) United States
(12) Patent Application Publication
(10) Pub. No.: US 2019/0006030 A1
(43) Pub. Date: Jan. 3, 2019

(54) AUTOMATED HEALTHCARE SYSTEM

(71) Applicant: Hill-Rom Services, Inc., Batesville, IN (US)

(72) Inventors: Matthew D. Mullin, Memphis, NY (US); Scott E. Cornell, Cazenovia, NY (US); David L. Kellner, Baldwinsville, NY (US); Kristin A. Alisanski, Syracuse, NY (US); Kenneth V. Coon, III, Jordan, NY (US); Robert J. Lawrence, Hudsonville, MI (US); Jack Barney Sing, Batesville, IN (US)

(21) Appl. No.: 16/019,865

(22) Filed: Jun. 27, 2018

Related U.S. Application Data
(60) Provisional application No. 62/526,138, filed on Jun. 28, 2017.

(51) Int. Cl. G16H 10/65; G16H 40/20; A61B 5/00

(52) U.S. Cl. CPC G16H 10/65; G16H 80/00; A61B 5/0022; G16H 40/20

(57) ABSTRACT

A system for monitoring a location of a patient in a healthcare facility may include a patient radiofrequency identification (RFID) tag, multiple wireless RFID readers disposed in multiple different locations in the healthcare facility, and a server computer configured to receive data from the multiple wireless RFID readers, determine the location of the patient in the healthcare facility and transmit the patient location data to a user computing device.

### RELATED APPLICATION(S)

This patent application claims the benefit of U.S. Patent Application Ser. No. 62/526,138 filed on Jun. 28, 2017, the entirety of which is hereby incorporated by reference.

### BACKGROUND

There are a number of different ways in which patient care and workflow management in hospitals, outpatient surgery centers and other healthcare environments are less efficient than they could be. For example, when a patient is undergoing surgery, there is currently no effective way to keep family members and friends apprised of the patient's progress and whereabouts. Typically, the patient's loved ones wait in a waiting room, and a nurse or physician must come out into the waiting room to update the loved ones about the patient's condition and progress. Not only is this inefficient, but it often leaves family and friends waiting for hours in the waiting room.

Other areas of inefficiency include the discharge process and workflow and movement of patients, healthcare providers and medical equipment within a healthcare institution. These inefficiencies can and do occur in any healthcare setting, such as hospitals, outpatient surgery centers and physician clinics.

### SUMMARY

In one aspect of the present disclosure, a system for monitoring a location of a patient in a healthcare facility includes a patient radiofrequency identification (RFID) tag configured to be worn by the patient, multiple wireless RFID readers disposed in multiple different locations in the healthcare facility, and a server computer configured to receive patient location data from the multiple wireless RFID readers, determine the location of the patient in the healthcare facility based on the patient location data, and transmit the location of the patient to a user computing device. In some embodiments, the system further includes a healthcare worker RFID tag configured to be worn by a healthcare worker, and the system is further configured to monitor a location of the healthcare worker in the healthcare facility.

In various embodiments, the system may further include the user computing device. In some embodiments, the user computing device includes an alarm device configured to alert the user when the patient has arrived at a predefined location in the healthcare facility. The predefined location, for example, may be a post-operative area where patients are taken after surgical procedures are completed. The user computing device may also include a display for displaying information about the patient. In some embodiments, the server computer is further configured to determine an estimated discharge time for the patient from the healthcare facility.

In another aspect of the present disclosure, a method for identifying a location of a patient in a healthcare facility may involve: attaching a patient radiofrequency identification (RFID) tag to the patient; reading the patient RFID tag with a wireless RFID reader disposed in the location in the healthcare facility; pairing the patient RFID tag with the location in the healthcare facility where the wireless RFID reader is located; and transmitting patient location data to a user computing device. Different locations in the healthcare facility where the RFID readers might be located include, but are not limited to, a post-operative area where patients wait after undergoing surgical procedures, a pre-operative area where patients wait before undergoing surgical procedures; and operating rooms where patients undergo surgical procedures.

In some embodiments, multiple RFID readers are disposed in multiple different locations in the healthcare facility, and the method involves monitoring patient movement from one of the different locations to another of the different locations. Optionally, the method may also involve monitoring a location of a healthcare worker in the healthcare facility. This may involve, for example, attaching a healthcare worker RIFD tag to the healthcare worker, reading the healthcare worker RFID tag with the multiple RFID readers, pairing the healthcare worker RFID tag with the different locations in the healthcare facility where the wireless RFID readers are located, and transmitting healthcare worker location data to a healthcare facility computing device. In some embodiments, the pairing and transmitting steps are performed by a processor on a server computer wirelessly connected with the wireless RFID reader. In some embodiments, the method may also involve comparing, with the processor, a movement of the healthcare worker in the healthcare facility with a predefined healthcare worker workflow.

In another aspect of the disclosure, a mobile medical sensor system for a healthcare facility includes a hub device comprising a reader and a processing unit and having a fixed location in the healthcare facility and multiple mobile medical sensors wirelessly connected to the hub device. In one embodiment, each of the mobile medical sensors is configured to automatically power on and collect data when it comes within range of the hub device. In one embodiment, each of the mobile medical sensors is configured to collect data in a mode that is consistent with the fixed location of the hub device in the healthcare facility. Each of the mobile medical sensors may also be configured to automatically send data to the hub device when it is within range of the hub device.

Some embodiments may also include a display coupled with the hub device, where the display is configured to automatically power on and display sensor information when one or more of the mobile medical sensors is within range of the hub device. For example, the display may be configured to automatically display information related to a patient and/or a clinician caring for the patient. In such embodiments, or in any other embodiments, the system may further include one or more RFID tags, configured to be worn by the patient and/or clinician. In some embodiments, the display is configured to automatically display a different set of information when the clinician is within range of the hub device than when the clinician is out of range of the hub device.

In some embodiments, the hub device is configured to aggregate information and automatically populate patient forms. In some embodiments, the hub device is configured to aggregate information and automatically send the information to an electronic medical record of a patient. In some embodiments, each of the mobile medical sensors includes an RFID tag.

In another aspect of the present disclosure, a method for sensing and providing patient health information includes: detecting the presence of a mobile medical sensor with a hub device comprising a reader and a processing unit and having a fixed location in the healthcare facility; automatically turning on the mobile medical sensor and sensing a patient parameter in a patient, in response to the mobile medical sensor coming within a predefined range of the hub device; and automatically sending data related to the sensed patient parameter from the mobile medical sensor to the hub device.

In one embodiment, the method also involves automatically powering on a display, in response to the data being sent to the hub device. Optionally, the method may also involve automatically displaying the data on the display. In one embodiment, automatically displaying the data involves displaying a different set of data when a clinician is within range of the hub device than when the clinician is out of range of the hub device. Such a method may further include identifying the clinician with the hub device by reading an RFID tag attached to the clinician with an RFID reader of the hub device. Some embodiments of the method also involve aggregating the data and automatically populating patient forms with the hub device. Some embodiments further involve aggregating the data and automatically sending the data to an electronic medical record of a patient.

In another aspect of the disclosure, a system for evaluating workflow in a healthcare facility includes: multiple radiofrequency identification (RFID) tags configured to be attached to at least one of healthcare workers or healthcare equipment in the healthcare facility; multiple wireless RFID readers disposed in multiple different locations in the healthcare facility; and a server computer configured to receive data from the multiple wireless RFID readers, determine the different locations of the multiple wireless RFID readers, and process the received data to generate workflow data related to movements of at least one of the healthcare workers or the healthcare equipment through the healthcare facility during a period of time. Again, examples of the multiple different locations include, but are not limited to a post-operative area, a pre-operative area and operating rooms. In some embodiments, the server computer is further configured to identify trends in the movements and/or bottlenecks in the movements.

In another aspect of the disclosure, a method for evaluating workflow in a healthcare facility involves: attaching multiple radiofrequency identification (RFID) tags to healthcare workers and/or healthcare equipment in the healthcare facility; reading the multiple RFID tags with multiple wireless RFID readers disposed in multiple different locations in the healthcare facility; receiving data from the multiple wireless RFID readers at a server computer; and generating workflow data from the received data. The workflow data is related to movements of the healthcare workers and/or the healthcare equipment through the healthcare facility during a period of time.

In another aspect of the disclosure, a method for facilitating discharge of a patient from a healthcare facility includes: receiving patient data describing at least one physiological parameter of the patient, acquired from at least one wireless sensor device, at a computer processor; processing the patient data, with the computer processor, to generate a patient discharge score for the patient; comparing the patient discharge score to a preset threshold discharge score, using the processor; determining, with the processor and based on the comparison of the patient discharge score to the threshold discharge score, that the patient is ready to be discharged from the healthcare facility; and transmitting, from the processor to a user computing device, a message indicating that the patient is ready to be discharged. In some embodiments, the method may also involve receiving, at the processor, the threshold discharge score from a clinician. In some embodiments, the method may also include receiving, at the processor, patient location information describing a location of the patient in the healthcare facility. The patient location information may be derived from an RFID tag attached to the patient and at least one RFID reader in the healthcare facility.

These and other aspects and embodiments are described in greater detail below, in relation to the attached drawings.

### DESCRIPTION OF THE DRAWINGS

FIG. 1 shows an example of an automated healthcare system for tracking patient locations in a healthcare facility.

FIG. 2 shows an example of a method using the system of FIG. 1.

FIG. 3 shows an example of an automated healthcare system for automatically controlling one or more sensor devices and/or tracking clinician and patient locations in a healthcare facility.

FIG. 4 shows example modules of one of the wireless sensor devices of FIG. 3.

FIG. 5 shows example modules of the hub of FIG. 3.

FIG. 6 shows example physical components of the hub of FIG. 3.

### DETAILED DESCRIPTION

The present disclosure is directed to an automated healthcare system, in which locations and movements of patients, physicians and/or medical equipment can be tracked. Tracked locations of patients, clinicians and/or equipment may be used to enhance efficiency in a healthcare setting, such as but not limited to an ambulatory surgery center or a hospital. Some examples and embodiments are discussed in this application in the context of ambulatory surgery centers (“ASC,” also known as outpatient surgery centers, same day surgery centers or “surgicenters”), which are healthcare facilities where surgical procedures are performed that do not require an overnight hospital stay. Although the surgicenter example is often used, the embodiments described in this disclosure are not limited to use in any particular healthcare environment.

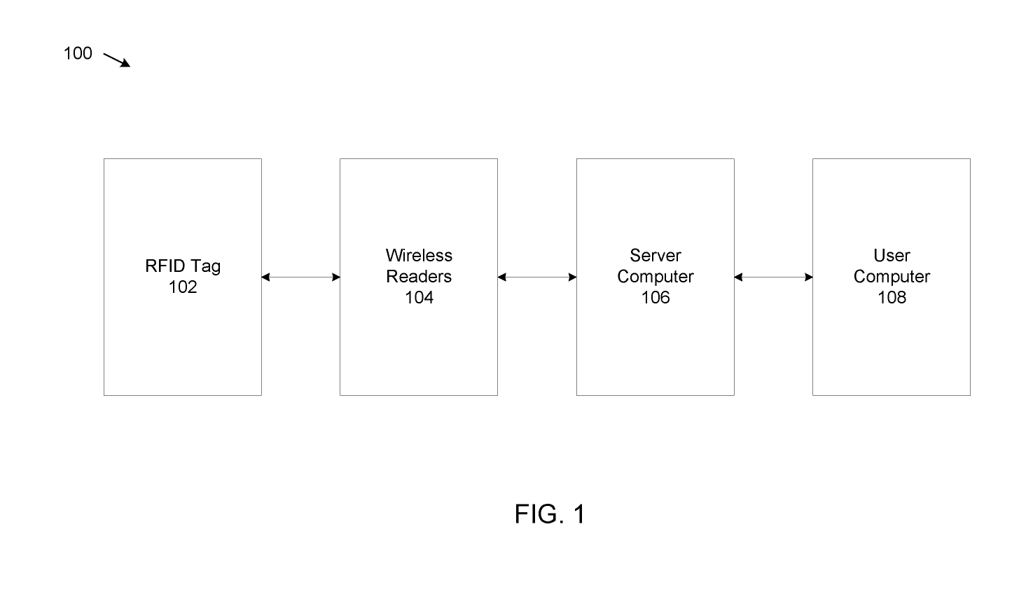

As mentioned above, one aspect of the present disclosure is a system and method for tracking a patient's location in a surgicenter, hospital or other healthcare setting. FIG. 1 shows an example patient tracking system 100, which may in some embodiments be part of an automated healthcare system. The system 100 includes a patient RFID (radiofrequency identification) tag 102, wireless readers 104, a server computer 106 and a user computer 108. In this embodiment, the patient RFID tag 102 is attached to the patient, for example via a wristband around the patient's wrist. As the patient is moved through the surgicenter, for example, the patient RFID tag 102 will be sensed by multiple wireless RFID readers 104 located throughout the surgicenter. For example, one set of wireless readers 104 may be located throughout a pre-operative area, a second set may be located in the operating rooms of the surgicenter, and a third set may be located throughout a post-operative area. In various embodiments, any number of wireless readers 104 may be disposed throughout any number of locations in a surgicenter, hospital or other healthcare delivery environment. The wireless readers 104 are usually (but not necessarily) stationary and include identifying information, identifying their locations within the healthcare environment. Examples of stationary objects to which the readers 104 might be attached include but are not limited to hospital beds, walls, door frames and stationary medical equipment.

The wireless readers 104 transmit signals to the server computer 106 when the readers 104 detect an RFID tag 102 of a patient. The server computer 106 then processes the signals to generate patient location information. For example, a processor on the server computer 106 may pair the identity of a sensed RFID tag 102 with a location of the wireless reader 104 that sensed the RFID tag 102. The processor thus can provide a location of a particular patient within the healthcare environment. The server computer 106 may be located in any suitable location, such as within the healthcare environment, outside of the healthcare environment, in the cloud, or the like. Once the server computer 106 has generated patent location data, it will then transmit the data to one or more user computers 108, informing the user(s) as to the whereabouts of the patient. In various embodiments, the wireless readers 104 may communicate wirelessly with the server computer 106 using any suitable wireless communication interface, and the server computer 106 may similarly communicate wirelessly with the user computer(s) 108 using any suitable wireless communication interface. Examples of wireless communication interfaces include, but are not limited to, Bluetooth, near field communication (NFC), ZigBee, ultra-wide-band, ultrasound, infrared, WIFI, and radio-frequency identification (RFID).

In one embodiment, the user may be a family member or friend of the patient, and the user computer 108 may be a smart phone with an application that receives the patient location information from the server computer 106. In some embodiments, there may be multiple user computers 108, such as a smart phone used by a family member and a desktop computer at a front desk of the surgicenter, a nurse's station at a hospital, or the like. Another example of a user computer 108 may be an alert “puck”—e.g., a small, mobile alert device, which friends, family or the like may hold and carry within or outside of the healthcare facility and which sounds an alarm, lights up, vibrates and/or otherwise indicates when the patient moves to a new location. For example, the puck may alert the user(s) when the patient moves out of the operating room and into a post-operative recovery area. This signal would allow the friends and family using the puck to know that the patient was close to a time for visitation and/or to return home.

In some embodiments, the server computer 106 may provide additional information to the user computer(s) 108 regarding a patient's status, in addition to the patient's location. For example, in some embodiments, the user computer 108, such as a smart phone with a specific application for the purpose, may be configured to inform the user that the patient has arrived at a particular milestone in a surgical procedure, that the patient has a certain amount of time remaining in the surgical procedure, that there is a certain amount of time remaining until the patient will be discharged from the healthcare facility, and/or the like. In some embodiments, all of this patient status information may be generated automatically, for example with a processor in the server computer 106 being configured to calculate approximate times until discharge, surgical procedure completion, etc. In other embodiments, some of the patient status information may be entered into the system 100 by a healthcare provider or staff. For example, in one embodiment, an operating room nurse may have access to one of the user computers 108 located in an operating room, and the nurse may be able to enter information about the patient's status, such as an approximate time remaining until completion of the surgical procedure. Similarly, a nurse in a post-operative area may have access to one of the user computers 108 and may be able to input information, such as an approximate time of discharge of a particular patient. These user computers 108 may be any suitable device, such as a desktop computer, a mobile tablet device, or even a smart phone, according to various embodiments.

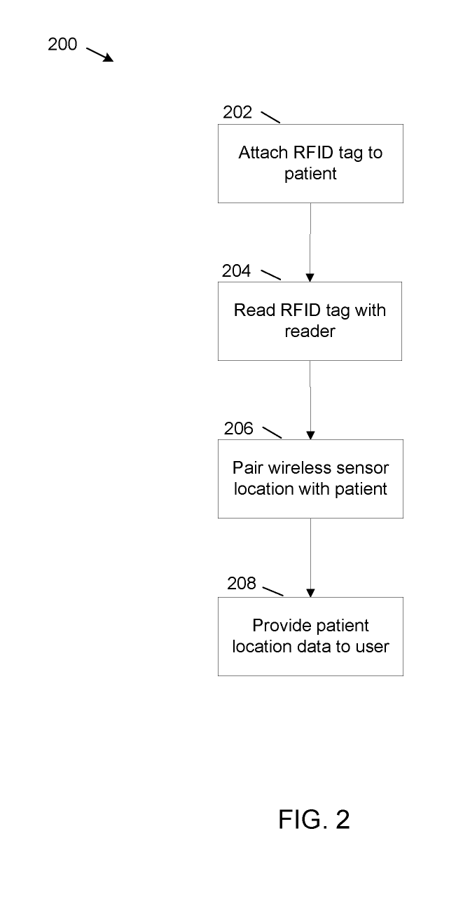

FIG. 2 illustrates a patient tracking method 200, using the system 100 just described. In this embodiment, the method 200 involves, at step 202, attaching an RFID tag 102 to the patient, as mentioned above. At step 204, the RFID tag 102 is sensed with one or more wireless readers 104, for example located in an operating room. A processor on a server computer 106 receives sensed signals from the wireless reader(s) 104 and, in step 206, it pairs a location of the readers 104 with the identity of the patient according to the RFID tag 102. Finally, in step 208, the server computer 106 provides patient location data to one or more users on one or more user computing devices 108, such as but not limited to a smart phone, a mobile tablet device, a desktop computer, a laptop computer or a puck alerting device. In alternative embodiments, the patient tracking method 200 may have any of a number of additional or alternative steps. For example, as mentioned above, in some embodiments one or more healthcare workers may input additional patient location or status information into the system 100 to provide additional information to the patient's family, friends or other users.

In some embodiments, the patient tracking system 100 and method 200 may be used for purposes other than (or in addition to) informing family members and friends of a patient's progress through the healthcare environment. For example, tracking multiple patients' locations during their stays in a hospital facility may allow a user to better understand whether patient flow through the facility is as efficient as desired and, if not, how efficiency improvements might be made. In an ASC setting, for example, a user might observe that patients tend to spend more time than necessary waiting in a pre-operative area of the surgicenter. Thus, the pre-op area might be identified as a workflow bottleneck. Similarly, patients might wait for longer periods of time than necessary in a post-op recovery area before being discharged. This might be another bottleneck. A surgicenter or other hospital might also be able to determine that patients are spending too much time in the operating rooms, for example if there is too much lag time between when patients are brought into the operating room and when the surgical procedures are started. This might be due, for example, to longer than desirable anesthesiology preparation time. In some embodiments, one of the users of the system 100 and method 200 may be an efficiency consultant or other similar expert, who may provide a service to an ASC, hospital or the like, to enhance efficiency of the healthcare facility. This type of use of the system 100 and method 200 for efficiency analysis is described further below, in relation to additional examples.

One specific use of the patient tracking system 100 and method 200 may be to facilitate patient discharge from an ASC, hospital or other healthcare facility. In many healthcare environments, the patient discharge process is highly inefficient and time consuming, which may be aggravating for the patient, as well as family and friends, and may also be costly for the healthcare facility, in terms of wasted hospital bed occupancy time. The patient tracking system 100 and method 200 may be used, in some environments, to enhance efficiency of the process. For example, a patient's location in the healthcare facility may be tracked, to help determine the patient's readiness for discharge. In one example, if the patient has gotten out of bed and walked down the hall a certain number of times per day, that level of activity might be one factor indicating that the patient is ready for discharge. If the patient's location in a surgery center indicates that she arrived in the post-operative recovery room one hour ago, that might be a factor indicative of readiness for discharge. In some embodiments, the processor on the server computer 106 may be configured to receive a number of different data points related to a patient and analyze the different data points to automatically determine whether and/or when the patient is ready for discharge.

Alternatively or in addition to the patient location information generated and used by the patient tracking system 100 and method 200, other patient information may be used to help determine a patient's readiness for discharge. For example, medical sensing equipment, such as vital signs monitors, may be used to measure any of a number of vital signs and/or other patient parameters. This information may be transmitted to the server computer 106, which may in turn use any or all of the received information about a given patient to automatically determine a time at which the patient can or should be discharged from the facility. A processor on the server computer 106 may, for example, automatically generate a “patient readiness score” or a “discharge readiness score,” based on a patient's vital signs, location, ability to ambulate, other information about the patient entered into the system by a nurse, physician or rehabilitation specialist, and/or the like. The patient readiness score may be provided to a user, or the server computer 106 may use the score to calculate a suggested time for the patient to be discharged. In one specific example, when a patient is transferred from an operating room to a post-op recovery area in an ASC, the patient's RFID tag 102 may be read by one or more wireless readers 104 located in the post-op area (on the bed, on the wall, etc.). A nurse or other healthcare provider may attach one or more sensing devices to the patient in the post-op area, for sensing vital signs.

In this embodiment, the patient's location and arrival time in the post-op area and the patient's sensed vital signs are all transmitted to the server computer 106. All (or alternatively only some) of this patient data may then be used by the server computer 106 to determine, and even track over time, the patient's readiness for discharge from the ASC.

In some embodiments, the server computer 106 may be configured to determine patient discharge readiness based on set criteria and patient readiness scores that are preset. In other embodiments, a physician or other healthcare worker may set a discharge readiness score for each patient, and the server computer 106 determines when the discharge score for each particular patient is met. In such an embodiment, for example, the server computer 106 may continuously receive vital signs information for a patient from one or more vital signs monitors, and it may continuously analyze the vital signs information to determine when/whether the criteria for meeting the set discharge score have been met. When the discharge score set by the healthcare provider for the patient is achieved or exceeded, the server computer 106 may provide an indicator, such as an alert or simply information, to one or more user computers 108, so that the healthcare providers will know that discharge is appropriate. In various embodiments, any suitable type and number of criteria may be used to determine a discharge score. Again, this is only one exemplary embodiment, and many other variations are possible.

In an alternative embodiment of the system 100 and method 200 to the one just described, one or more RFID tags 102 may be worn by any number of healthcare workers and/or may be attached to any number and type of mobile healthcare equipment. To be clear, various embodiments may include RFID tagging any number of (1) patients, (2) healthcare workers, (3) mobile medical equipment, or (4) any combination thereof. Healthcare workers include, but are not limited to, physicians, nurses, physician assistants, medical technicians, orderlies and/or any other staff of a healthcare facility. Mobile medical equipment includes any equipment that might be moved from one location in the healthcare facility to another, such as but not limited to hospital beds, patient monitoring equipment, mobile computing devices, and physiological measurement devices, such as blood pressure cuffs, thermometers, pulse oximetry, ECG monitors, and the like. The locations and movements of healthcare workers and/or mobile medical equipment may be tracked, using the system 100 and method 200 described above, for any suitable purpose. For example, in some embodiments, locations of healthcare workers and equipment might be tracked so that they can easily be found immediately by other healthcare workers when needed. Additionally, the tracking system 100 and method 200 may be used over a period of time to identify trends. These trends may be used to identify bottlenecks and monitor, analyze and improve workflow and efficiency, as described above. Some or all of this analysis may be performed by a processor on the server computer 106, or alternatively it might be performed by a different computing device that receives data from the patient tracking system 100. In many embodiments, the server computer 106 may receive, collect and process incoming data from multiple wireless readers 104 throughout the healthcare facility, which are receiving signals from multiple RFID tags 102 attached to healthcare workers, medical equipment and/or patients. After processing the data, the server computer 106 may send the processed data on to one or more user computers 108. Again, as mentioned above, in some embodiments, one of the users of the system 100 and method 200 may be a service provider that helps an ASC, hospital or other facility evaluate and improve efficiency.

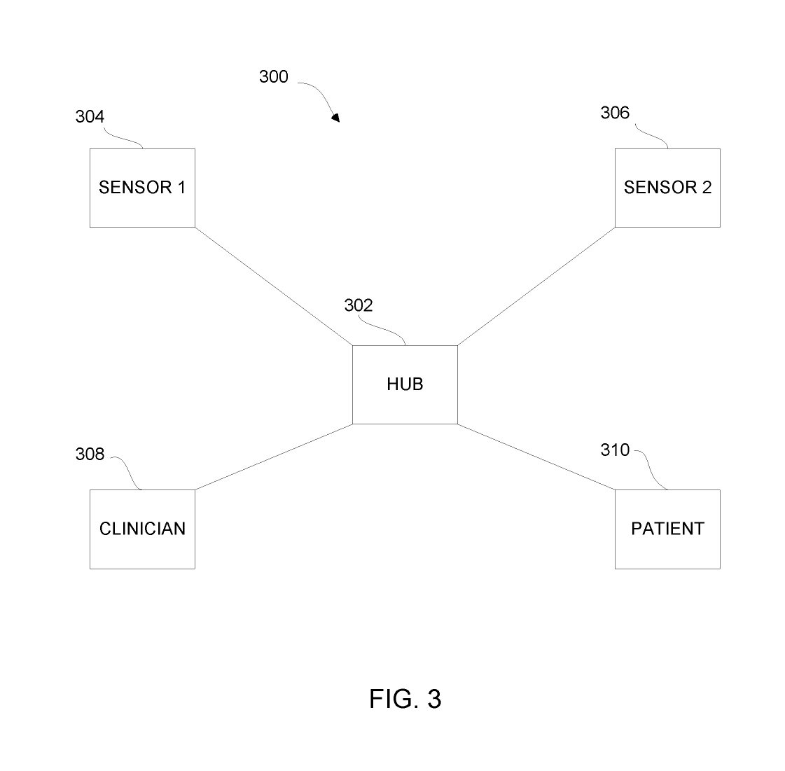

FIG. 3 shows an embodiment of an automated healthcare system 300. In some embodiments, the patient tracking system 100 of FIG. 1 may be a part of the automated healthcare system 300, but this is not required. In this embodiment, the system 300 includes a hub 302, a first wireless sensor 304, a second wireless sensor 306, a clinician 308 and a patient 310. These latter components of the system 300—the first wireless sensor 304, the second wireless sensor 306, the clinician 308 and the patient 310—are only provided here for exemplary purposes, and in alternative embodiments the system may include only one sensor 304 or any number of additional sensors, and it may or may not include the clinician 308 and/or the patient 310, as will be explained further below. In a simplest embodiment, the system 300 may include only the hub 302 and one of the other components shown in FIG. 3. In other embodiments, many additional components may be included. Thus, the embodiment described here is only one embodiment and is not intended to limit the scope of the present disclosure.

The hub 302 is a piece of permanent (or “fixed” or “non-mobile”) medical equipment or furniture located in an ambulatory surgery center, hospital or other healthcare environment. The example of the hub 302 that will be used here is a hospital bed, but this is only one example. The hub 302 acts as a communication center or “system coordinator” for the automated healthcare system 300, and it sends and receives information to one or more of the other system components. It has an awareness of its location—for example, a hospital bed hub 302 knows that it is located in a pre-op area, an operating room, a post-op area, a patient room, or the like. The hub 302 may communicate with one or more wireless sensors 304, 306 whenever the sensors 304, 306 are moved within a certain range of the hub 302. Whether a sensor 304, 306 is within range of the hub 302 may typically be determined using a form of near field communication, for example using Bluetooth between sensor 304, 306 is within range of the hub 302. The sensors 304, 306 may include, for example, any number and type of vital signs monitors, ECG monitors, and the like. Wireless communication interfaces that may be used by the hub 302 and the sensors 304, 306 include, but are not limited to, Bluetooth, near field communication (NFC), ZigBee, ultra-wide-band, ultrasound, infrared, WIFI, and radio-frequency identification (RFID). In some embodiments, any sensor device, such as the first wireless sensor 304, the second wireless sensor 306 and/or any other device in a surgicenter or hospital, which moves within range of the hub 302 will be able to communicate with the hub 302.

In one embodiment, one or more of the sensors 304, 306 may automatically act in a particular way when it comes within range of the hub 302. For example, in a simplest embodiment, the sensors 304, 306 may simply turn on automatically when they come within range of the hub 302. In some embodiments, the sensors 304, 306 may turn on and also start sensing patient data (vital signs, for example), when they come within range of the hub 302. In a further level of detail, some or all of the sensors 304, 306 may communicate with the hub 302 to determine the location of the hub 302 within the healthcare facility, and the sensors 304, 306 may operate in a specific way, based on the location. For example, the first wireless sensor 304 may be a vital signs sensor. If the first wireless sensor 304 is moved into a pre-operative area that is within range of the hub 302, it will receive a signal from the hub 302, identifying the location as the pre-operative area. In response to this location signal, the first wireless sensor 304 may begin sensing patient vital signs in a manner that is typically desired for that location, such as a spot-check (or “one-time”) manner for the pre-op area. If, on the other hand, the first wireless sensor 304 is moved into an operating room and comes within range of the hub 302 in that room, the first wireless sensor 304 may start sensing patient vital signs in an ongoing fashion—e.g., a “monitoring mode” or continuously or at preset intervals. If the hub 302 were located instead in a post-op recovery area of an ASC or hospital, the first wireless sensor 304 may sense vital signs of the patient in the spot-check fashion, as in the pre-op area, or in some different mode. Additionally, any information sensed by the sensors 304, 306 may be sent automatically to the hub 302, as long as the sensors 304, 306 are in range of the hub 302. A display on the hub 302 and/or displays on the sensors 304, 306 may also automatically power on and display information when the sensors 304, 306 come within range of the hub 302. Therefore, the automated healthcare system 300 can be used to automatically control any number of wireless patient sensor devices 304, 306 and collect any number and type of patient data, as the sensors 304, 306 move into and out of range of the hub 302.

In some embodiments, the hub 302 may also include an RFID reader component, which can read RFID tags worn by one or more clinicians 308 and/or the patient 310. The clinician may be a physician, nurse, physician's assistant or any other healthcare provider or staff of the healthcare facility. In various embodiments, as the clinician 308 comes within range of the hub 302, the hub 302 may perform a specific function or operate in a way that is specific to the presence of the clinician 308. For example, in some embodiments, the hub 302 may be a hospital bed with a display monitor attached to it, such as a television or computer monitor. The patient 310 may watch various entertainment items on the display or may alternatively use the display as a computer monitor, for example controlling it with a touch screen, remote control or wireless keyboard. When the clinician 308 comes into the patient's room and within range of the hub 302, whatever is currently being displayed on the monitor may change to a clinician-specific display. For example, the patient's medical record, vital signs, discharge information and/or any other suitable patient information may be displayed on the monitor, for review by the clinician 308 and possibly also the patient 310. In some embodiments, the hub 302 may also function in different ways based on whether the patient 310 is present. For example, the hub 302 may turn on a display when the patient 310 is in bed and may turn the display off when the patient 310 leaves the bed and is out of range of the hub. Thus, the automated healthcare system 300 may operate to automatically control one or more sensors 304, 306, as well as the hub 302, based on the presence of the sensors 304, 306, the clinician(s) 308 and/or the patient 310 within range of the hub 302.

In addition to the functions described above, the automated healthcare system 300 may also be configured to provide additional functions. For example, in some embodiments, the reader in the hub 302 may collect data from the sensors 304, 306, the clinician RFID tag 308 and/or the patient RFID tag 310, aggregate the data, and use the data to perform certain functions, such as but not limited to automatically populating healthcare forms or transmitting some or all of the collected data to the patient's electronic medical record. In a “fully automated” embodiment of the automated healthcare system, all healthcare workers and all patients in a facility might wear RFID tags, and all wireless sensing devices and other mobile devices would be tagged and/or otherwise capable of registering or communicating with the hub 302. In this embodiment, movements and locations of equipment, patients and clinicians could be closely tracked, and trends could be identified and evaluated. This type of monitoring and evaluation could be used to make changes in the healthcare facility to enhance efficiency and patient care.

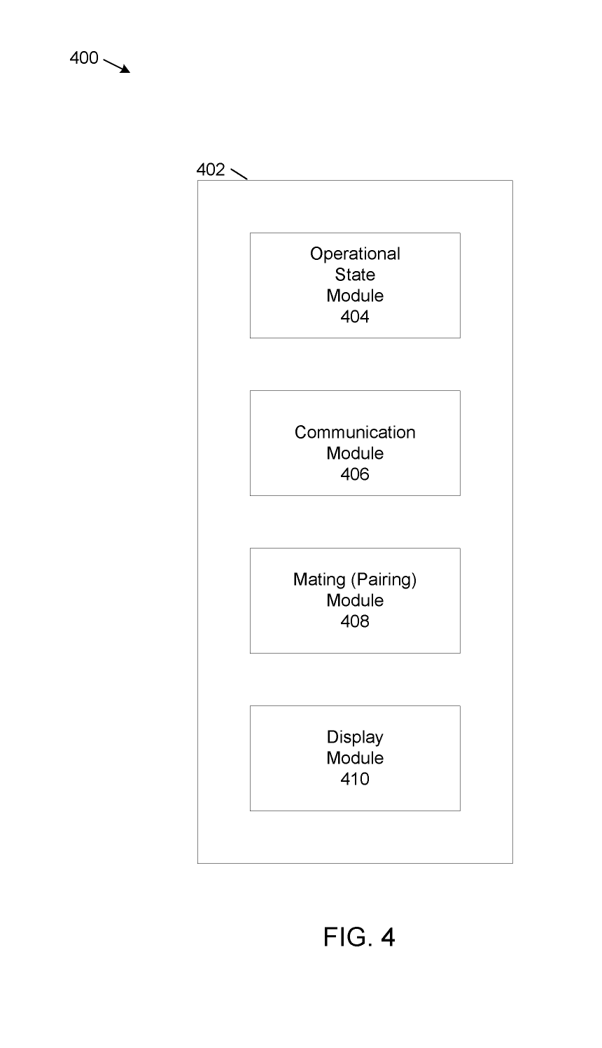

FIG. 4 shows example modules 400 of a wireless sensor device 402, which may be an example of one of the sensor devices 304, 306 described above. The example wireless sensor device 402 includes an operational state module 404, a communication module 406, a mating (pairing) module 408 and a display module 410.

The example operational state module 404 monitors and stores status of operational states that comprise a patient parameter measurement process using wireless sensor device 402. Example states in the patient parameter measurement process include powering on the wireless sensor device 402 when in range of a hub 302 and taking a vital signs measurement of a patient. Other states are possible.

The example communication module 406 provides a communication interface for the wireless sensor device 402 for communicating with the wireless reader of the hub 302. In some examples, the communication interface supports Bluetooth. In other examples, the communication interface supports communication protocols like ZigBee, ultra-wide-band and others. The communication interface, as implemented in the communication module 406, permits data and control information to be communicated between the wireless sensor device 402 and the hub 302. The communication interface also permits a determination to be made of a distance between wireless sensor device 402 and the hub 302.

The example mating module 408 implements an electronic mating between wireless sensor device 402 and the hub 302. The electronic mating associates an identifier with wireless sensor device 402 and an identifier for the hub 302. In some examples, the identifier for wireless sensor device 402 may be displayed on a display device built into wireless sensor device 402. In other examples, wireless sensor device 402 may have a connector, for example a universal serial bus (USB) connector, in lieu of a display device. In some examples, the mating module 408 sends the identifiers for wireless sensor device 402 and the hub 302 to the server computer 106. The server computer 106 permits the mating identification information to be displayed on a client computer, as explained later herein. In other examples, the server computer 106 is not required. In these examples, the wireless sensor device 402 may be directly connected to the client computer 108 or to the hub 302, and the identifiers may be displayed on the client computer or on the hub 302.

The example display module 410 permits a display of information on a display device of wireless sensor devices 402 that have a display device. In some examples, the information may include an identifier for the wireless sensor device 402. In some examples, the information may also include an identifier and location for the hub 302. In other examples, the information may also include a most recent vital sign measurement for a patient. In some examples, the wireless sensor device 402 may not include a display device or a display module 410.

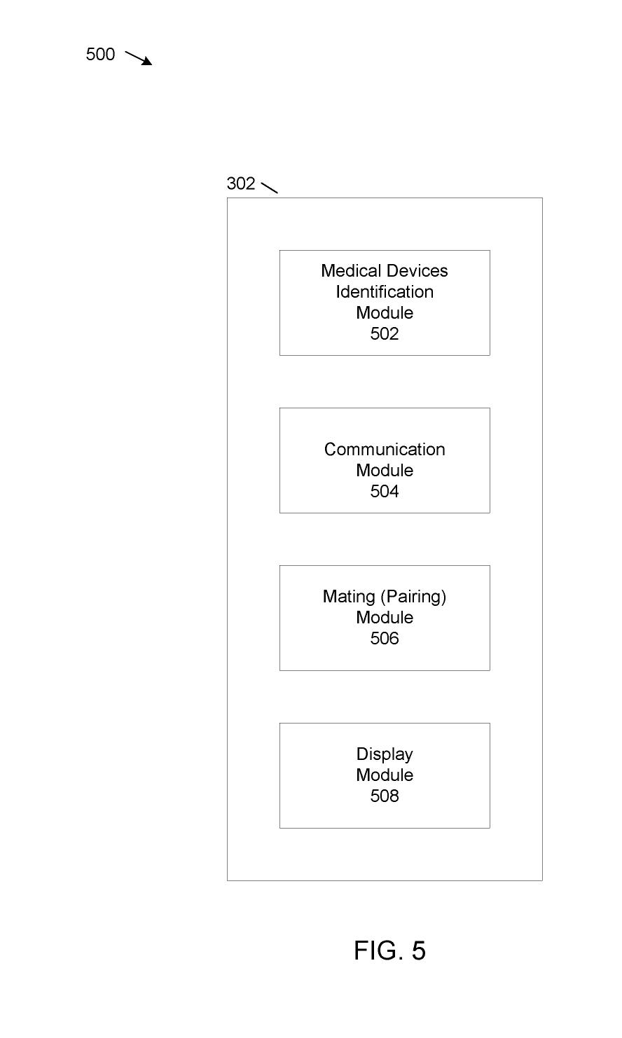

FIG. 5 shows example modules 500 of the hub 302. The example modules 500 include a medical devices identification module 502, a communication module 504, a mating module 506 and a display module 508.

The example medical devices identification module 502 stores identifiers for each wireless sensor device 402, which may include blood pressure cuffs, oxygen saturation sensors, thermometer probes, ECG sensors, etc. The example communication module 504 provides a communication interface for the hub 302 for communicating with wireless sensor device 402. In some examples, the communication interface supports Bluetooth. In other examples, the communication interface supports communication protocols like ZigBee, ultra-wide-band and others. The communication interface as implemented in the communication module 504 permits data and control information to be communicated between the hub 302 and the wireless sensor device 402. The communication interface also permits a determination to be made of a distance between the hub 302 and the wireless sensor device 402.

The example mating module 506 implements an electronic mating between the wireless sensor device 402 and the hub 302. The electronic mating associates an identifier with the wireless sensor device 402 and an identifier for the hub 302. The mating module 506 sends the identifiers for the wireless sensor device 402 and the hub 302 to the server computer 106. The server computer 106 permits the mating identification information to be displayed on a client computer 108. As discussed earlier, in some examples the server computer 106 is not required. In these examples, the wireless sensor device 402 may be directly connected to the client computer 108 or to the hub 302 to display the mating identification information.

The example display module 508 permits a display of information on the hub 302. The information may include identifiers for one or more of the handheld medical devices associated with the hub 302. The information may also include mating information for the wireless sensor device 402 that may be mated to the hub 302. In addition, the information may include current measurement data from the wireless sensor device 402, such as blood pressure readings, temperature, oxygen saturation, heart rate, etc.

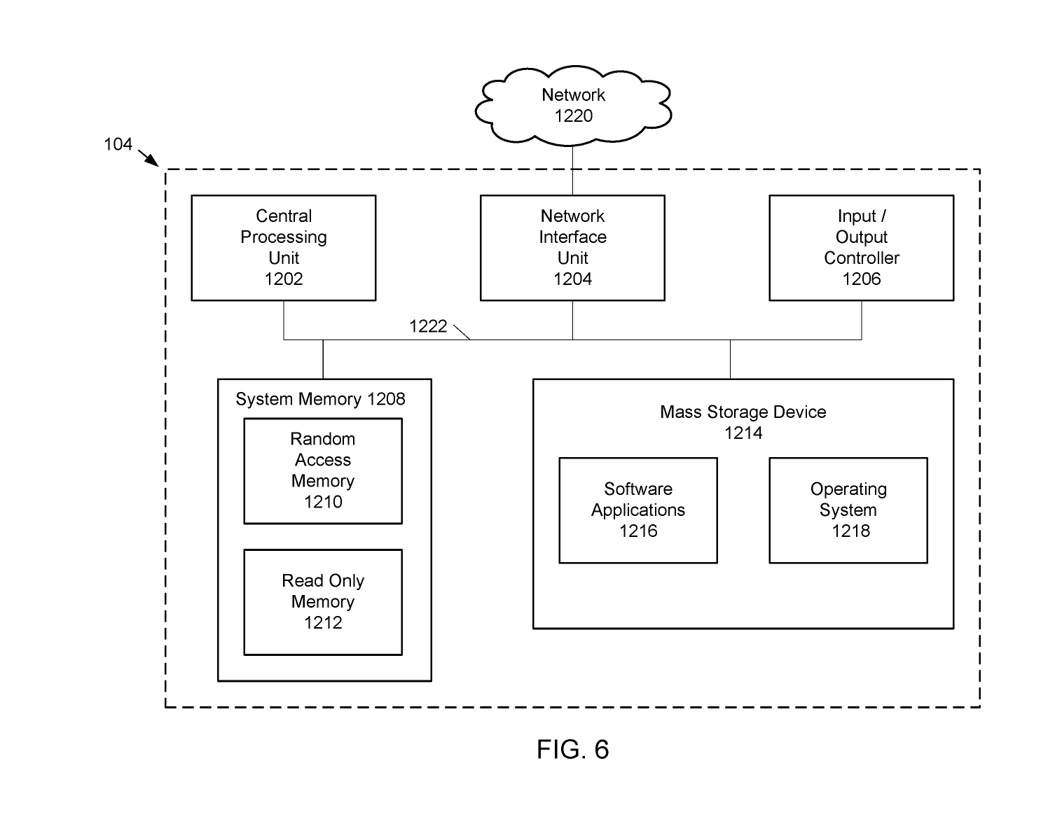

FIG. 6 illustrates example physical components of the hub 302. As illustrated in the example of FIG. 6, the hub 302 includes at least one central processing unit (“CPU”) 1202, a system memory 1208, and a system bus 1222 that couples the system memory 1208 to the CPU 1202. The system memory 1208 includes a random access memory (“RAM”) 1210 and a read-only memory (“ROM”) 1212. A basic input/output system contains the basic routines that help to transfer information between elements within the hub 302, such as during startup, is stored in the ROM 1212. The hub 302 further includes a mass storage device 1214. The mass storage device 1214 is able to store software instructions and data.

The mass storage device 1214 is connected to the CPU 1202 through a mass storage controller (not shown) connected to the bus 1222. The mass storage device 1214 and its associated computer-readable data storage media provide non-volatile, non-transitory storage for the hub 302. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the hub 302.

According to various embodiments of the invention, the hub 302 may operate in a networked environment using logical connections to remote network devices through the network 1220, such as a local network, the Internet, or another type of network. The hub 302 may connect to the network 1220 through a network interface unit 1204 connected to the bus 1222. It should be appreciated that the network interface unit 1204 may also be utilized to connect to other types of networks and remote computing systems. The hub 302 also includes an input/output controller 1206 for receiving and processing input from a number of other devices, including a keyboard, a mouse, a touch user interface display screen, or another type of input device. Similarly, the input/output controller 1206 may provide output to a touch user interface display screen, a printer, or other type of output device.

As mentioned briefly above, the mass storage device 1214 and the RAM 1210 of the hub 302 can store software instructions and data. The software instructions include an operating system 1218 suitable for controlling the operation of the hub 302. The mass storage device 1214 and/or the RAM 1210 also store software instructions, that when executed by the CPU 1202, cause the hub 302 to provide the functionality of the hub 302 discussed in this document. For example, the mass storage device 1214 and/or the RAM 1210 can store software instructions that, when executed by the CPU 1202, cause the hub 302 to display received physiological data on the display screen of the hub 302.

The physical components shown in FIG. 6 may also apply to one or more patient wireless sensor device 304, 306, 402. For example, the physical components shown in FIG. 6 may also apply to a wireless thermometer probe, an oxygen saturation sensor, etc.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A system for monitoring a location of a patient in a healthcare facility, the system comprising:
a patient radiofrequency identification (RFID) tag configured to be worn by the patient;
multiple wireless RFID readers disposed in multiple different locations in the healthcare facility; and
a server computer configured to receive patient location data from the multiple wireless RFID readers, determine the location of the patient in the healthcare facility based on the patient location data, and transmit the location of the patient to a user computing device.

2. The system of claim 1, further comprising a healthcare worker RFID tag configured to be worn by a healthcare worker, wherein the system is further configured to monitor a location of the healthcare worker in the healthcare facility.

3. The system of claim 1, wherein the system further comprises the user computing device.

4. The system of claim 3, wherein the user computing device comprises an alarm device configured to alert the user when the patient has arrived at a predefined location in the healthcare facility.

5. The system of claim 4, wherein the predefined location comprises a post-operative area where patients are taken after surgical procedures are completed.

6. The system of claim 3, wherein the user computing device comprises a display for displaying information about the patient.

7. The system of claim 1, wherein the server computer is further configured to determine an estimated discharge time for the patient from the healthcare facility.

8. A method for identifying a location of a patient in a healthcare facility, the method comprising:
attaching a patient radiofrequency identification (RFID) tag to the patient;
reading the patient RFID tag with a wireless RFID reader disposed in the location in the healthcare facility;
pairing the patient RFID tag with the location in the healthcare facility where the wireless RFID reader is located; and
transmitting patient location data to a user computing device.

9. The method of claim 8, wherein the location in the healthcare facility comprises a post-operative area where patients wait after undergoing surgical procedures.

10. The method of claim 9, wherein multiple RFID readers are disposed in multiple different locations in the healthcare facility, and wherein the multiple locations further comprise:
a pre-operative area where patients wait before undergoing surgical procedures; and
operating rooms where patients undergo surgical procedures.

11. The method of claim 8, wherein multiple RFID readers are disposed in multiple different locations in the healthcare facility, and wherein the method comprises monitoring patient movement from one of the different locations to another of the different locations.

12. The method of claim 11, further comprising monitoring a location of a healthcare worker in the healthcare facility, wherein monitoring the location of the healthcare worker comprises:
attaching a healthcare worker RIFD tag to the healthcare worker;
reading the healthcare worker RFID tag with the multiple RFID readers;
pairing the healthcare worker RFID tag with the different locations in the healthcare facility where the wireless RFID readers are located; and
transmitting healthcare worker location data to a healthcare facility computing device.

13. The method of claim 8, wherein the pairing and transmitting steps are performed by a processor on a server computer wirelessly connected with the wireless RFID reader.

14. The method of claim 13, further comprising comparing, with the processor, a movement of the healthcare worker in the healthcare facility with a predefined healthcare worker workflow.

15. A system for evaluating workflow in a healthcare facility, the system comprising:
multiple radiofrequency identification (RFID) tags configured to be attached to at least one of healthcare workers or healthcare equipment in the healthcare facility;
multiple wireless RFID readers disposed in multiple different locations in the healthcare facility; and
a server computer configured to receive data from the multiple wireless RFID readers, determine the different locations of the multiple wireless RFID readers, and process the received data to generate workflow data related to movements of at least one of the healthcare workers or the healthcare equipment through the healthcare facility during a period of time.

16. The system of claim 15, wherein the multiple different locations comprise:
a post-operative area where patients wait after undergoing surgical procedures.
a pre-operative area where patients wait before undergoing surgical procedures; and
operating rooms where patients undergo surgical procedures.

17. The system of claim 16, wherein the server computer is further configured to identify at least one of trends in the movements or bottlenecks in the movements.

18. The system of claim 15, wherein the server computer is further configured to identify at least one of trends in the movements or bottlenecks in the movements.

19. The system of claim 15, wherein the server computer further monitors patient movement through the healthcare facility.

20. The system of claim 15, wherein the server computer further pairs the multiple RFID tags with the different locations in the healthcare facility where the wireless RFID readers are located.
